(19) United States
(12) Patent Application Publication
(10) Pub. No.: US 2024/0116840 A1
(43) Pub. Date: Apr. 11, 2024

(54) NOVEL TRIS(ALLYL ETHER) COMPOUND HAVING TRIPHENYLALKANE BACKBONE

(71) Applicant: HONSHU CHEMICAL INDUSTRY CO., LTD., Chuo-ku, Tokyo (JP)

(72) Inventors: Akihito NASU, Wakayama-shi (JP); Kazuhisa YAJIMA, Wakayama-shi (JP); Takeru YOSHII, Wakayama-shi (JP); Masafumi OTSUKI, Wakayama-shi (JP); Kentaro YAMANE, Wakayama-shi (JP)

(21) Appl. No.: 18/262,908

(22) PCT Filed: Feb. 10, 2022
(86) PCT No.: PCT/JP2022/005243

§ 371 Date: Jul. 25, 2023

(30) Foreign Application Priority Data
Feb. 18, 2021 (JP) 2021-024275

(51) Int. Cl. C07C 43/215

(52) U.S. Cl. CPC C07C 43/215

(57) ABSTRACT

An object is to provide a compound that can be a novel curing agent. The compound suppresses volatilization of the curing agent that can lead to a decrease in function of a prepreg, contamination of equipment for producing the prepreg, and in addition a decrease in production yield of the prepreg, and further contributes to improving dielectric properties. As a solution, a tris(allyl ether) compound represented by general formula (1) below is provided.

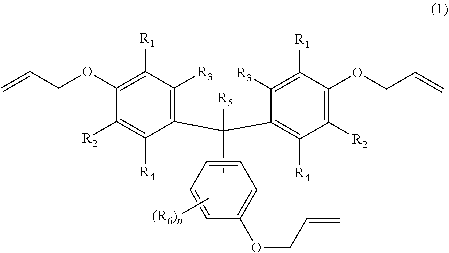

### TECHNICAL FIELD

The present invention relates to a novel tris(allyl ether) compound having a triphenylalkane backbone, the compound being less likely to volatilize when used as a curing agent and contributing to improving dielectric properties.

### BACKGROUND ART

As curing agents for polyphenylene ether resins and thermosetting resins such as epoxy resins and bismaleimide resins and as crosslinking agents for polyolefin resins and the like, compounds having an allyl group are used, and in particular triallyl isocyanurate is widely used.

In a situation where a thermosetting resin forming a prepreg is cured, heating to a high temperature of about 200° C. is performed, and thus there have been the following problems: triallyl isocyanurate, which is liquid at normal temperature (25° C.), readily volatilizes together with a solvent, and the decrease in the amount of curing agent to cross-link with the thermosetting resin results in a cured resin with degraded properties; the volatilized curing agent contaminates production equipment, and it takes time and effort to clean the equipment; the yield of the product decreases; and so on.

PTL 1 describes an example where a compound having two or more allyloxyphenyl moieties is used for a thermosetting resin composition containing an epoxy compound, the composition being used as a solventless adhesive, a sealing material for electric and electronic components, a paint, or the like.

In recent years, in electronic device applications, where thermosetting resins are used as materials, adaptability to higher frequencies has been required, and materials with low relative dielectric constants and low dielectric loss tangents that enable reductions in transmission loss have been required.

The present inventors have paid attention to “1,1,1-tris(4-allyloxyphenyl)ethane” and “1,1,1-tris(4-allyloxyphenyl)methane” known in the art, which have a plurality of allyl groups and have a highly heat resistant triphenylalkane as a basic backbone. However, it has been confirmed that both the compounds have higher heat resistance than triallyl isocyanurate but their dielectric properties are not satisfactory.

### CITATION LIST

### Patent Literature

PTL 1: Japanese Unexamined Patent Application Publication No. 06-136095

### SUMMARY OF INVENTION

### Technical Problem

The present invention has been made in view of the foregoing circumstances, and an object thereof is to provide a compound that can be a novel curing agent. The compound suppresses volatilization of the curing agent that can lead to a decrease in function of a prepreg, contamination of equipment for producing the prepreg, and in addition a decrease in production yield of the prepreg, and further contributes to improving dielectric properties.

### Solution to Problem

To achieve the above object, the present inventors have conducted intensive studies and found that by introducing a substituent at the ortho position of an allyloxy group on a benzene ring of a tris(allyl ether) compound having a triphenylalkane backbone known in the art, the formation of a hydroxy group due to a Claisen rearrangement during heating can be prevented, and thus excellent dielectric properties are exhibited while high heat resistance is maintained, thereby completing the present invention.

The present invention is as follows.

1. A tris(allyl ether) compound represented by general formula (1) below.

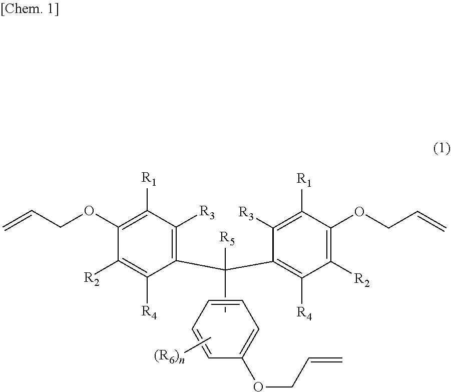

(In the formula, R₁ and R₆ each independently represent a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms, R2, R3, and R₄ each independently represent a hydrogen atom, a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms, R₅ represents a hydrogen atom or a linear or branched alkyl group having 1 to 6 carbon atoms, and n represents 0 or an integer of 1 to 4.)
2. The tris(allyl ether) compound according to 1, represented by general formula (2) below.

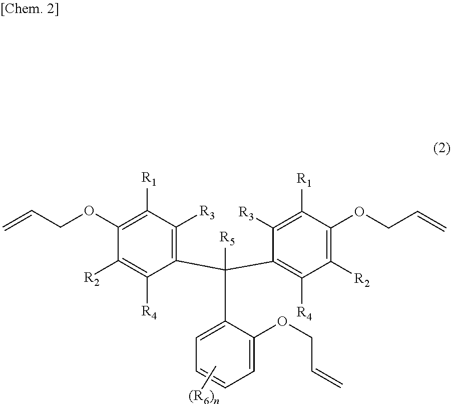

(In the formula, R₁ to R₆ and n are as defined in general formula (1).)
3. The tris(allyl ether) compound according to 1, represented by general formula (3) below.

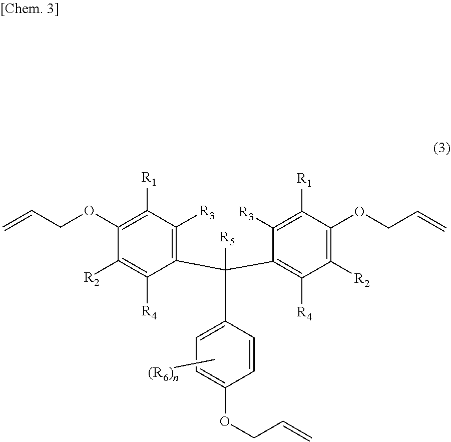

(In the formula, R₁ to R₆ and n are as defined in general formula (1).)

### Advantageous Effects of Invention

The tris(allyl ether) compound according to the present invention, when used as a curing agent or a crosslinking agent, produces excellent effects, that is, suppression of volatilization due to high heat resistance and contribution to improving dielectric properties of the material used.

The novel compound according to the present invention can be suitably used in the electrical and electronic components field, where high reliability is required, particularly as a curing agent for epoxy resins, bismaleimide resins, and polyphenylene ether resins, which are raw materials for semiconductor sealing materials, printed wiring boards, build-up wiring boards, solder resists, and the like, and can also be suitably used as a crosslinking agent for polyolefin resins and the like and a raw material for polyglycidyloxy compounds.

### DESCRIPTION OF EMBODIMENTS

Hereinafter, the present invention will be described in detail.

A compound according to the present invention includes tris(allyl ether) compounds represented by general formulae (1) to (3) below.

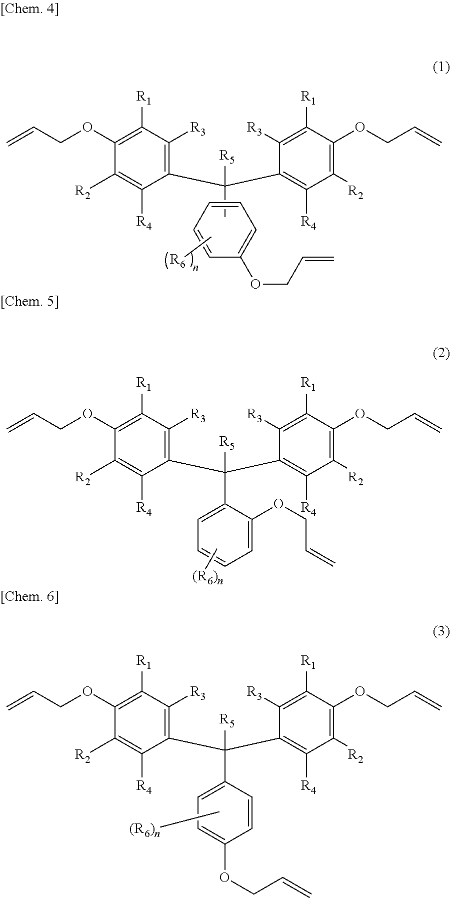

R₁ and R₆ in general formulae (1) to (3) each independently represent a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms. In particular, R₁ and R₆ are each preferably any of linear or branched alkyl groups having 1 to 4 carbon atoms, cyclic alkyl groups having 5 to 6 carbon atoms, linear or branched alkoxy groups having 1 to 4 carbon atoms, and cyclic alkoxy groups having 5 to 6 carbon atoms, more preferably any of linear or branched alkyl groups having 1 to 4 carbon atoms and cyclic alkyl groups having 5 to 6 carbon atoms, still more preferably any of linear or branched alkyl groups having 1 to 4 carbon atoms, particularly preferably an alkyl group having 1 carbon atom, that is, a methyl group.

R₂ in general formulae (1) to (3) each independently represent a hydrogen atom, a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms. In particular, R₂ is preferably any of a hydrogen atom, linear or branched alkyl groups having 1 to 4 carbon atoms, cyclic alkyl groups having 5 to 6 carbon atoms, linear or branched alkoxy groups having 1 to 4 carbon atoms, and cyclic alkoxy groups having 5 to 6 carbon atoms, more preferably any of a hydrogen atom, linear or branched alkyl groups having 1 to 4 carbon atoms, and cyclic alkyl groups having 5 to 6 carbon atoms, still more preferably any of linear or branched alkyl groups having 1 to 4 carbon atoms, particularly preferably an alkyl group having 1 carbon atom, that is, a methyl group.

R₃ and R₄ in general formulae (1) to (3) each independently represent a hydrogen atom, a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms. In particular, R₃ and R₄ are each preferably any of a hydrogen atom, linear or branched alkyl groups having 1 to 4 carbon atoms, cyclic alkyl groups having 5 to 6 carbon atoms, linear or branched alkoxy groups having 1 to 4 carbon atoms, and cyclic alkoxy groups having 5 to 6 carbon atoms, more preferably any of a hydrogen atom, linear or branched alkyl groups having 1 to 4 carbon atoms, and cyclic alkyl groups having 5 to 6 carbon atoms, still more preferably any of a hydrogen atom and linear or branched alkyl groups having 1 to 4 carbon atoms, particularly preferably a hydrogen atom or an alkyl group having 1 carbon atom, that is, a hydrogen atom or a methyl group.

In general formulae (1) to (3), both or one of R₃ and R₄ on the same benzene ring is preferably a hydrogen atom.

R₅ in general formula (1) represents a hydrogen atom or a linear or branched alkyl group having 1 to 6 carbon atoms. In particular, R₅ is preferably any of a hydrogen atom and linear or branched alkyl groups having 1 to 4 carbon atoms, more preferably any of a hydrogen atom and linear or branched alkyl groups having 1 to 4 carbon atoms, particularly preferably a hydrogen atom or an alkyl group having 1 carbon atom, that is, a hydrogen atom or a methyl group. When R₅ is a hydrogen atom, it is advantageous in that the starting material is relatively easily synthesizable and available. When R₅ is a linear or branched alkyl group having 1 to 6 carbon atoms, compared to when R₅ is a hydrogen atom, it is advantageous in that volatilization is suppressed because of high heat resistance, low Claisen rearrangement reactivity of allyl groups upon heating and a small change in refractive index upon heating contribute to improving dielectric properties, and a compound with a good hue is easily obtained.

In particular, R₅ in general formula (2) is preferably any of a hydrogen atom and linear or branched alkyl groups having 1 to 4 carbon atoms, more preferably a hydrogen atom or an alkyl group having 1 carbon atom, that is, a hydrogen atom or a methyl group, particularly preferably a hydrogen atom. When R₅ is a hydrogen atom, it is advantageous in that the starting material is relatively easily synthesizable and available.

In particular, R₅ in general formula (3) is preferably any of a hydrogen atom and linear or branched alkyl groups having 1 to 4 carbon atoms, more preferably any of linear or branched alkyl groups having 1 to 4 carbon atoms, particularly preferably an alkyl group having 1 carbon atom, that is, a methyl group. When R₅ is a linear or branched alkyl group having 1 to 6 carbon atoms, compared to when R₅ is a hydrogen atom, it is advantageous in that volatilization is suppressed because of high heat resistance, low Claisen rearrangement reactivity of allyl groups upon heating and a small change in refractive index upon heating contribute to improving dielectric properties, and a compound with a good hue is easily obtained.

n in general formulae (1) to (3) represents 0 or an integer of 1 to 4.

Among the tris(allyl ether) compounds represented by general formula (1) according to the present invention, the tris(allyl ether) compound represented by general formula (2) or (3) above is preferred.

In general formula (2), n is preferably 0 or an integer of 1 to 3, more preferably 0 or an integer of 1 to 2, and is preferably 0 or 1. When n is not 0, at least one R₆ is preferably bonded to the ortho position with respect to the allyloxy group. Specific examples of structures in the case where n is not 0 include compounds (p-7) to (p-12) given later.

In general formula (3), n is preferably 0 or an integer of 1 to 3, more preferably an integer of 1 to 3, and is preferably 2 or 3. When n is not 0, at least one R₆ is preferably bonded to the ortho position with respect to the allyloxy group. Specific examples of structures in the case where n is not 0 include compounds (p-19) to (p-30) given later.

Specific examples of the tris(allyl ether) compound represented by general formula (1) include compounds (p-1) to (p-30) having the following chemical structures, and these are preferred. Compounds (p-1) to (p-12) are specific examples of the tris(allyl ether) compound represented by general formula (2), and compounds (p-13) to (p-30) are specific examples of the tris(allyl ether) compound represented by general formula (3).

Of these, compounds (p-1) to (p-3), compounds (p-5) to (p-9), compounds (p-11) and (p-12), compounds (p-14) to (p-18), and compounds (p-20) to (p-30) are more preferred, compounds (p-1) to (p-3), compounds (p-7) to (p-9), compounds (p-16) to (p-18), compounds (p-22) to (p-24), and compounds (p-28) to (p-30) are still more preferred, and compound (p-2), compound (p-3), compound (p-8), compound (p-9), compound (p-17), compound (p-18), compound (p-23), compound (p-24), compound (p-29), and compound (p-30) are particularly preferred.

That is, as the tris(allyl ether) compound represented by general formula (2), compounds (p-1) to (p-3), compounds (p-5) to (p-9) and compounds (p-11) and (p-12) are more preferred, compounds (p-1) to (p-3) and compounds (p-7) to (p-9) are still more preferred, and compound (p-2), compound (p-3), compound (p-8), and compound (p-9) are particularly preferred.

As the tris(allyl ether) compound represented by general formula (3), compounds (p-14) to (p-18) and compounds (p-20) to (p-30) are more preferred, compounds (p-16) to (p-18), compounds (p-22) to (p-24), and compounds (p-28) to (p-30) are still more preferred, and compound (p-17), compound (p-18), compound (p-23), compound (p-24), compound (p-29), and compound (p-30) are particularly preferred.

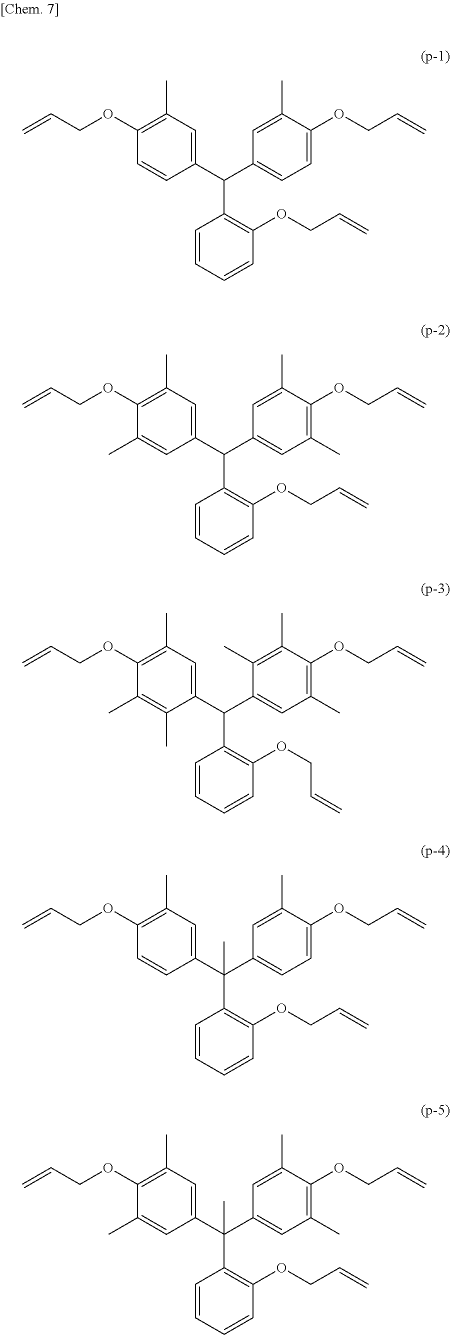

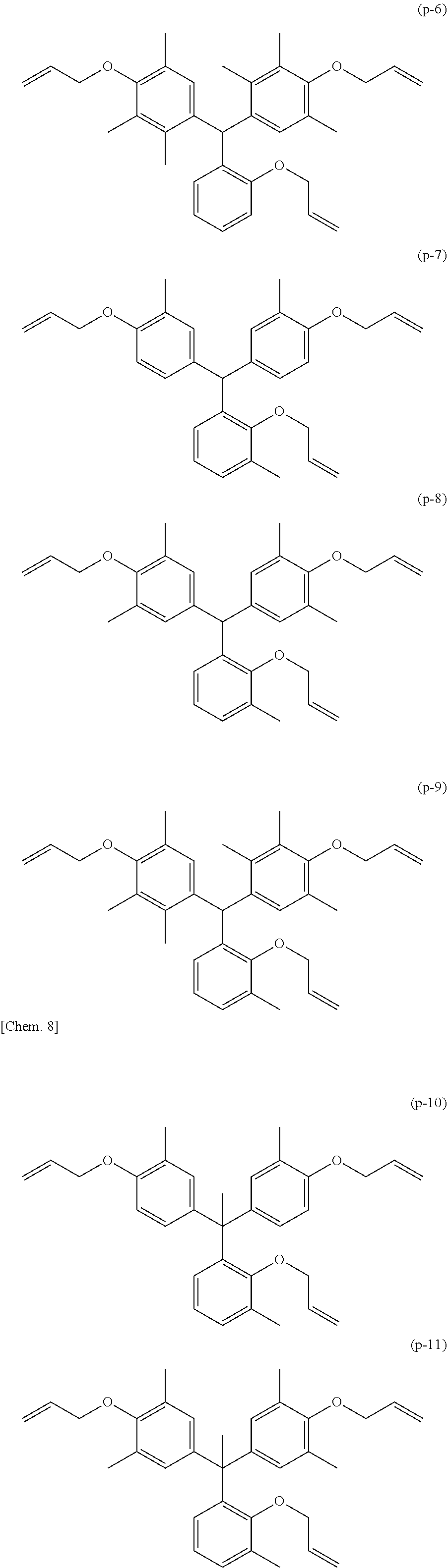

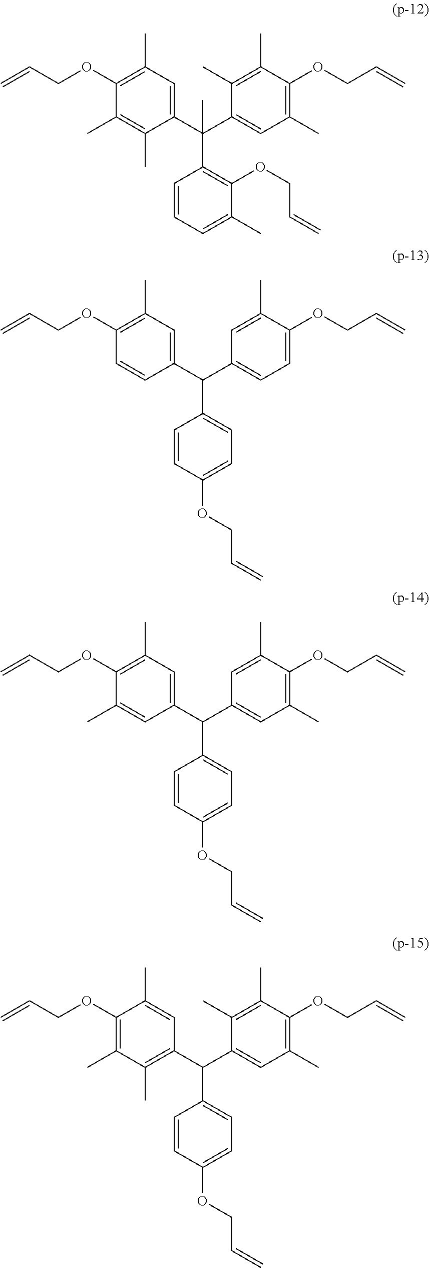

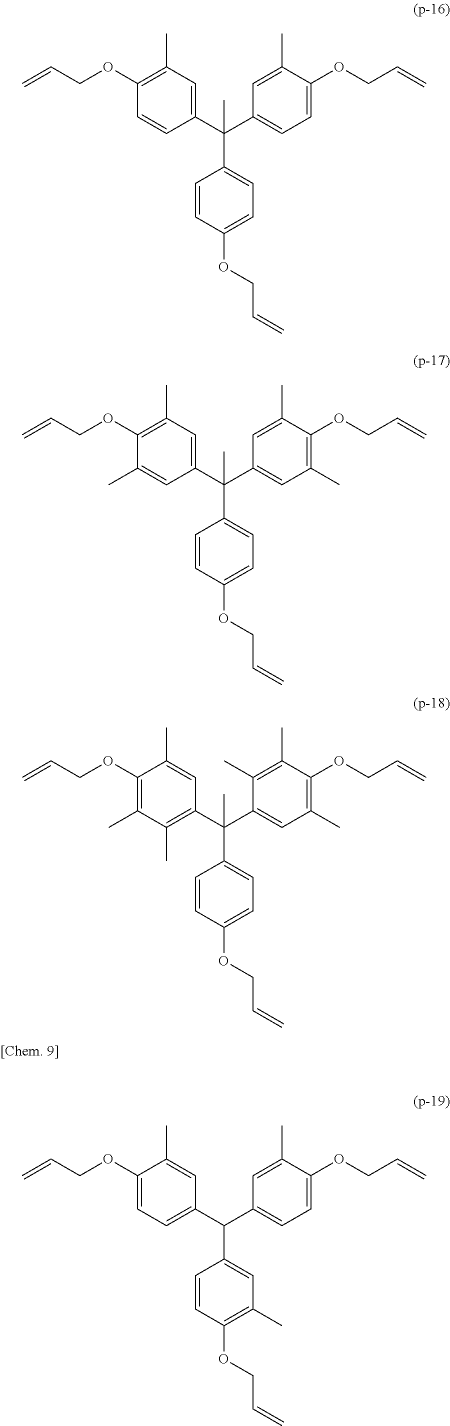

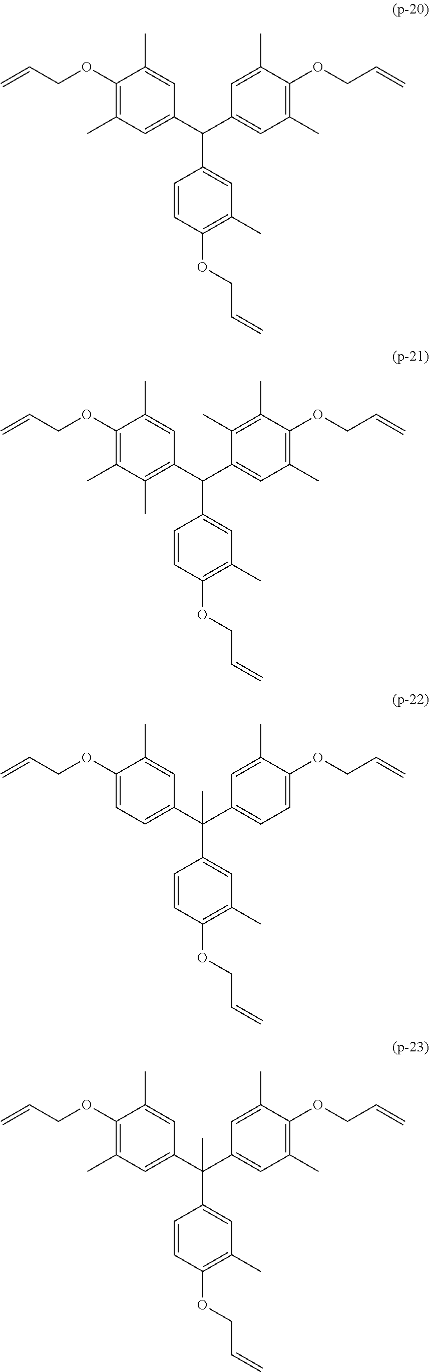

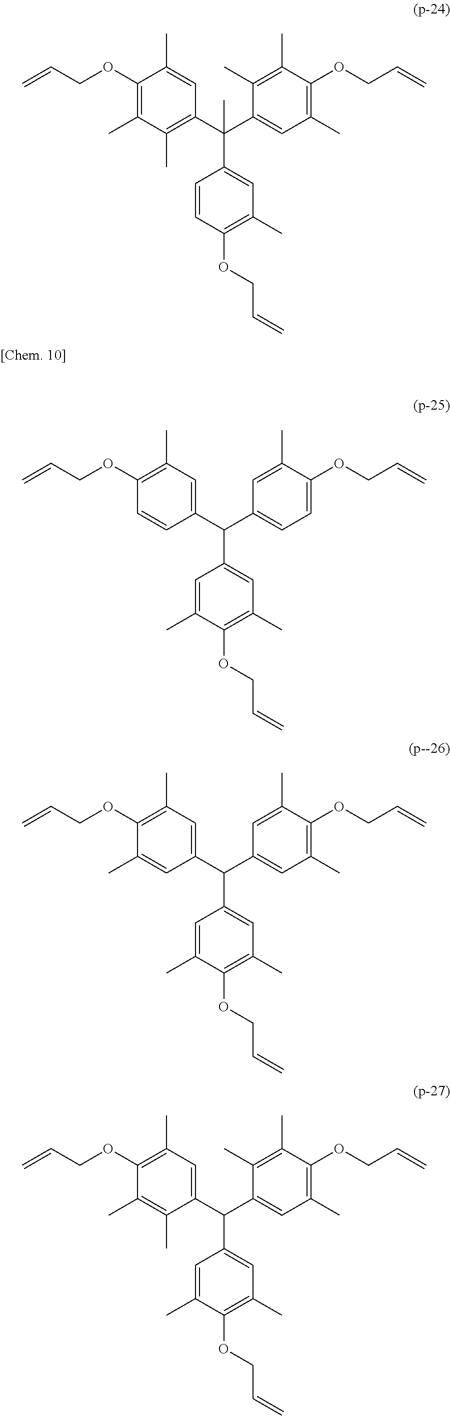

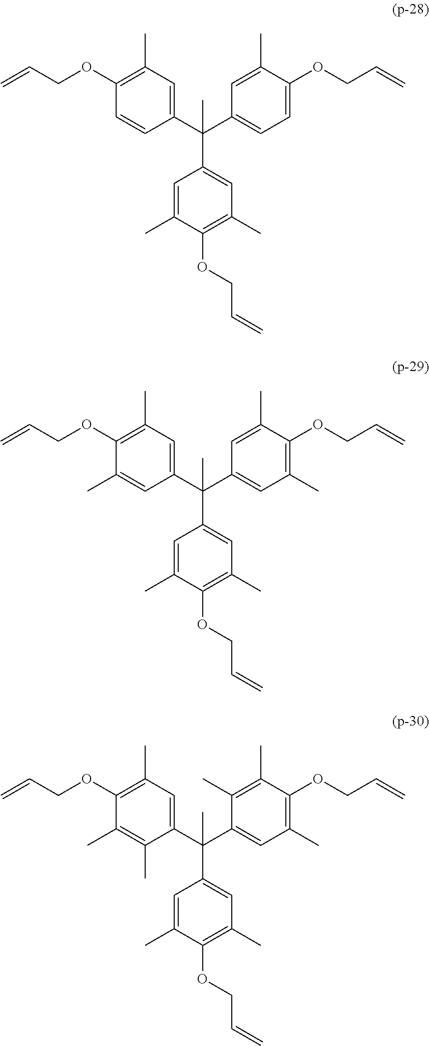

### <Method for Producing Inventive Compound>

For the tris(allyl ether) compound represented by general formula (1) according to the present invention, there are no particular limitations on the starting materials in the production of the tris(allyl ether) compound and the method for producing the tris(allyl ether) compound. For example, the target tris(allyl ether) compound represented by general formula (1) may be produced by allowing a trisphenol represented by general formula (4) and an allyl halide represented by general formula (5) to undergo a condensation reaction in the presence of a basic catalyst, as illustrated by the following reaction formula.

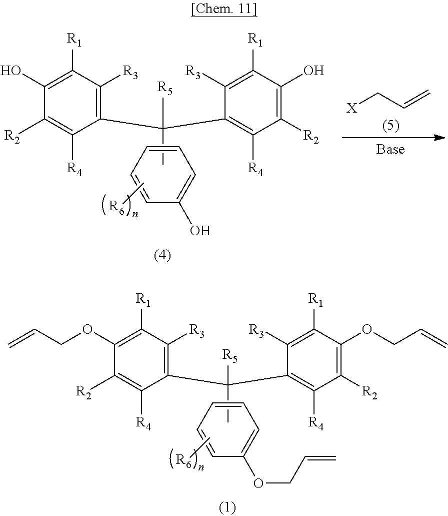

(In the formula, R₁ to R₆ and n are respectively the same as those in general formula (1), and X represents a halogen atom.)

### (Starting Material)

In the above production method, the trisphenol represented by general formula (4) used as a starting material is not particularly limited, and a trisphenol obtained by a known production method (e.g., a method described in Japanese Unexamined Patent Application Publication No. 06-107577, Japanese Unexamined Patent Application Publication No. 09-176068, or the like) can be used.

The allyl halide represented by general formula (5) used as a starting material is not particularly limited. Typically, allyl chloride, allyl bromide, allyl iodide, or the like is preferably used, and in particular allyl chloride or allyl bromide is preferably used.

In the above production method, the amount of the allyl halide represented by general formula (5) used is preferably in the range of 1 to 10 equivalents, more preferably in the range of 1 to 5 equivalents, still more preferably in the range of 1 to 3 equivalents, for one hydroxy group of the trisphenol represented by general formula (4).

### (Basic Catalyst)

In the above production method, the reaction is preferably performed in the presence of a basic catalyst in order to capture hydrogen halides formed. Examples of basic catalysts that can be used include inorganic bases such as sodium hydride, sodium carbonate, potassium carbonate, sodium hydroxide, and potassium hydroxide, and if necessary the reaction may be performed in the presence of a co-catalyst such as an alkali metal bromide, e.g., sodium bromide or potassium bromide, an alkali metal iodide, e.g., sodium iodide or potassium iodide, ammonium bromide, or ammonium iodide. These basic catalysts and co-catalysts are non-limiting examples.

In the above production method, the amount of basic catalyst used is preferably in the range of 1 to 10 equivalents, more preferably in the range of 1 to 5 equivalents, still more preferably in the range of 1 to 3 equivalents, for one hydroxy group of the trisphenol represented by general formula (4).

### (Reaction Conditions)

The reaction is typically performed in the presence of a solvent. The solvent is not particularly limited as long as it does not inhibit the reaction, and examples include alcohols such as methyl alcohol, ethyl alcohol, n-propyl alcohol, isopropyl alcohol, n-butyl alcohol, isobutyl alcohol, and sec-butyl alcohol; cyclic alkanes such as cyclopentane, cyclohexane, and cycloheptane; ethers such as diethyl ether, diisopropyl ether, tetrahydrofuran, and dioxane; ketones such as acetone, diethyl ketone, methyl-n-butyl ketone, and methyl isobutyl ketone; esters such as ethyl acetate, n-propyl acetate, isopropyl acetate, n-butyl acetate, and isobutyl acetate; nitriles such as acetonitrile; and amides such as N,N-dimethylformamide and N-methylpyrrolidone. These solvents can be used alone or in combination. The amount of solvent used is not particularly limited as long as the reaction is not hindered, and is typically in the range of 0.5 to 5 times, preferably in the range of 1 to 3 times the amount of the trisphenol represented by general formula (4) on a weight basis.

The reaction temperature is typically in the range of 0° C. to 120° C., preferably in the range of 10° C. to 80° C., more preferably in the range of 20° C. to 50° C. If the reaction temperature is excessively high, by-products increase to lower the yield, whereas if the reaction temperature is excessively low, the reaction rate slows down.

The reaction may be performed under normal pressure conditions or may be performed under increased pressure or reduced pressure.

### (Process After Completion of Reaction)

From the final reaction mixture obtained, the target tris(allyl ether) compound represented by general formula (1) can be obtained by a known method after completion of the reaction. For example, after the reaction, the remaining raw materials and solvent may be distilled off from the reaction mixture to thereby obtain the target as a residual liquid.

### EXAMPLES

The present invention will now be described specifically with reference to Examples, but it should be noted that the present invention is not limited to these Examples.

### <Analysis Method>

### 1. Purity Analysis (Analysis Values are Expressed in Area Percentage)

Measuring apparatus: Prominence UFLC high-performance liquid chromatography analyzer (manufactured by Shimadzu Corporation)

Pump: LC-20AD

Column oven: CTO-20A

Detector: SPD-20A

Column: HALO-C18 (inner diameter, 3 mm; length, 75 mm)

Oven temperature: 50° C.

Flow rate: 0.7 mL/min

Mobile phase: (A) 0.1 vol % aqueous acetic acid solution, (B) methanol

Gradient conditions: (A) vol % (time from start of analysis), 50% (0 min)→100% (7.5 min)→100% (20 min)

Sample injection volume: 5 μL

Detection wavelength: 280 nm

### 2. Hue (APHA)

After the following measuring instrument was calibrated using acetone, the dissolution color of a 2 wt % solution of a tris(allyl ether) compound in acetone was measured.

Measuring instrument: TZ6000 manufactured by Nippon Denshoku Industries Co., Ltd.

### Example 1: Synthesis of Compound (p-3)

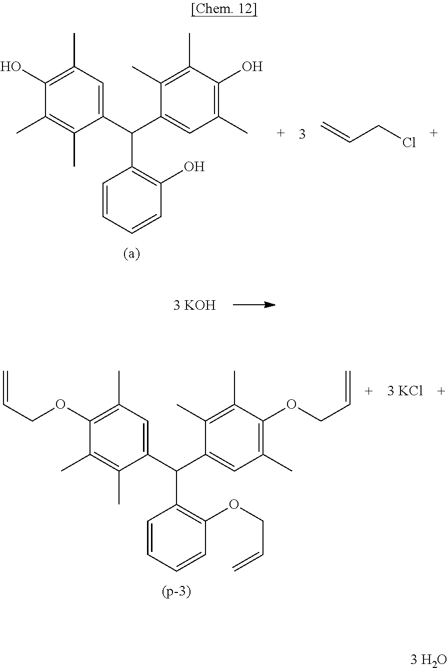

In a four-necked flask, 73.2 g of allyl chloride, 57.9 g of potassium hydroxide, and 50.0 g of acetone were loaded, and while maintaining the temperature at 38° C. to 42° C., a solution of 109.5 g of compound (a) in 200.0 g of acetone was slowly added dropwise. After completion of the dropwise addition, the mixture was stirred for 8 hours at the same reaction temperature. Thereafter, 198.0 g of water was added, and the reaction temperature was raised to 50° C. After stirring and standing, the aqueous layer was removed. Subsequently, 132.0 g of toluene and 132.0 g of water were added, and the mixture was stirred and left to stand, after which the aqueous layer was removed. An operation of adding 132.0 g of water to the oil layer obtained, stirring the mixture, and removing the aqueous layer after standing was repeated twice, thereby removing inorganic salts. The solvent in the obtained oil layer was then removed by reduced-pressure distillation to obtain an oily substance of compound (p-3).

The results of mass spectrometry and 1H-NMR analysis confirmed that compound (p-3), the target, was obtained.

Molecular weight of compound (p-3) (mass spectrometry/electrospray ionization): 519.3 (M+Na)⁺

¹H-NMR (400 MHZ, CDCl₃/TMS): δ2.00 (s, 6H), 2.12 (s, 6H), 2.19 (s, 6H), 4.25 (dd, J=1.4, 1.4 Hz, 4H), 4.39 (dd, J=1.6, 1.6 Hz, 2H), 5.10 (m, 2H), 5.23 (dd, J=1.2, 2.9 Hz, 2H), 5.42 (dd, J=1.6, 3.4 Hz, 2H), 5.77 (m, 1H), 6.00 (s, 1H), 6.10 (m, 2H), 6.38 (s, 2H), 6.70 (dd, J=1.4, 7.7 Hz, 1H), 6.83 (m, 2H).

### Comparative Example: Comparative Compounds 1 and 2

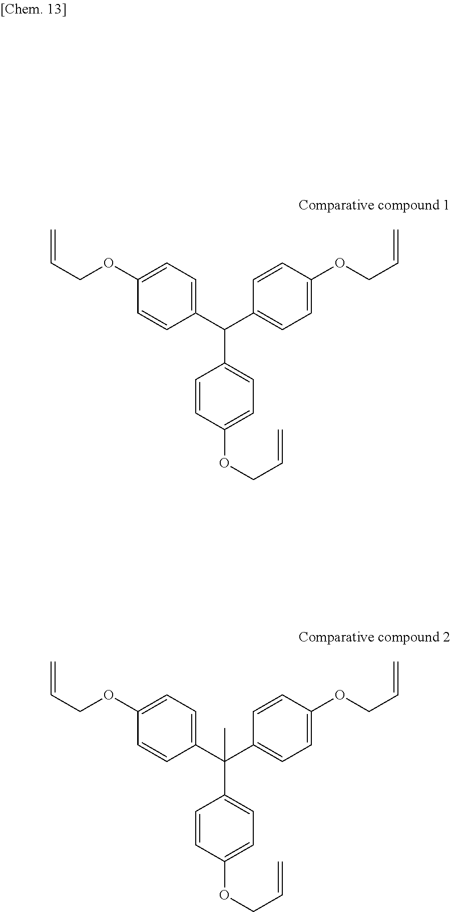

Comparative compound 1 and comparative compound 2 above were synthesized with reference to production methods described in Japanese Unexamined Patent Application Publication No. 2012-122011 and Japanese Unexamined Patent Application Publication (Translation of PCT Application) No. 2008-512524. Both the compounds were oily substances.

### Example 3: Synthesis of Compound (p-14)

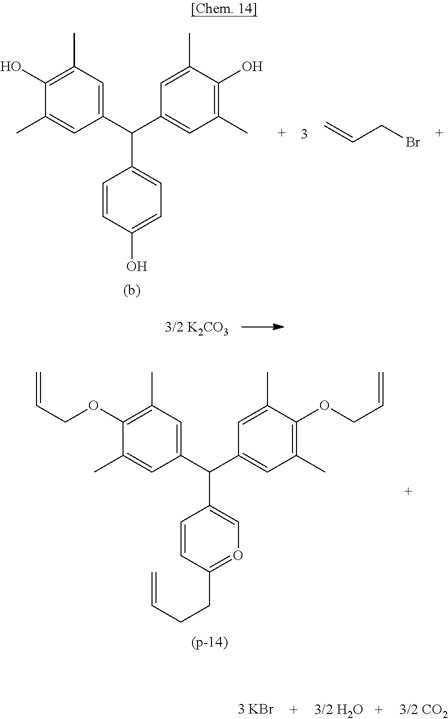

In a four-necked flask, 15.2 g of compound (b), 20.9 g of potassium carbonate, and 76.0 g of acetone were loaded, and while maintaining the temperature at 38° C. to 42° C., 18.5 g of allyl bromide was slowly added dropwise. After completion of the dropwise addition, the mixture was stirred at the same reaction temperature for 22 hours. Thereafter, 9.0 g of potassium carbonate and 5.8 g of allyl bromide were additionally added, and the mixture was further stirred for 23 hours. After completion of the reaction, filtration was performed, and the solid was washed with 15.0 g of acetone. Next, 20.4 g of water was added to the filtrate, and the reaction temperature was raised to 40° C. After stirring and standing, the aqueous layer was removed. Subsequently, 20.4 g of water and 63.4 g of butyl acetate were added, and the mixture was stirred and left to stand, after which the aqueous layer was removed. The solvent in the obtained oil layer was then removed by reduced-pressure distillation, and the concentrated oil was allowed to stand at room temperature for 2 days. After the standing, precipitated crystals were filtered, washed with 13.8 g of methanol, and then dried to obtain a yellow powder of compound (p-14).

The results of mass spectrometry and ¹H-NMR analysis confirmed that compound (p-14), the target, was obtained in a purity, as determined by high-performance liquid chromatography, of 97.4%. Compound (p-14) obtained had a hue (APHA) of 590.

Molecular weight of compound (p-14) (mass spectrometry/electrospray ionization): 467.3 (M−H)⁻

¹H-NMR (400 MHZ, CDCl₃/TMS): δ2.21 (s, 12H), 4.29 (dd, J=1.4, 1.4 Hz, 4H), 4.51 (dd, J=1.4, 1.4 Hz, 2H), 5.26 (m, 4H), 5.42 (m, 3H), 6.10 (m, 3H), 6.71 (s, 4H), 6.83 (d, J=8.4 Hz, 2H), 7.00 (d, J=8.4 Hz, 2H).

Example 3: Synthesis of compound (p-17)

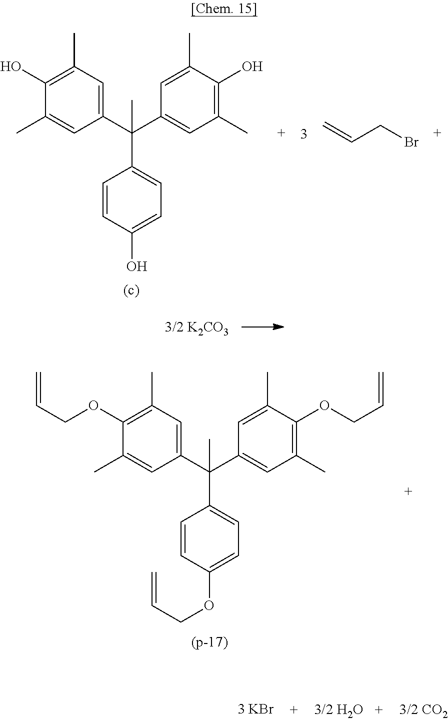

In a four-necked flask, 15.0 g of compound (c), 23.0 g of potassium carbonate, and 74.0 g of acetone were loaded, and while maintaining the temperature at 38° C. to 42° C., 20.0 g of allyl bromide was slowly added dropwise. After completion of the dropwise addition, the mixture was stirred at the same reaction temperature for 22 hours. Thereafter, 2.9 g of potassium carbonate and 2.4 g of allyl bromide were additionally added, and the mixture was further stirred for 24 hours. After completion of the reaction, filtration was performed, and washing was performed with 15.0 g of acetone. The solvent in the obtained oil layer was removed by reduced-pressure distillation, and 20.4 g of water and 20.4 g of butyl acetate were then added. The temperature was raised to 40° C., and after stirring and standing, the aqueous layer was removed. Subsequently, 20.4 g of water was added, and the mixture was stirred and left to stand, after which the aqueous layer was removed. The solvent in the obtained oil layer was then removed by reduced-pressure distillation, and the concentrated oil was allowed to stand at room temperature for 1 day. After the standing, precipitated crystals were filtered, washed with 17.7 g of methanol, and then dried to obtain a white powder of compound (p-17).

The results of mass spectrometry and ¹H-NMR analysis confirmed that compound (p-17), the target, was obtained in a purity, as determined by high-performance liquid chromatography, of 97.9%. Compound (p-17) obtained had a hue (APHA) of 40 and was shown to have an extremely good hue.

Molecular weight of compound (p-17) (mass spectrometry/electrospray ionization): 481.3 (M−H)⁻

¹H-NMR (400 MHz, CDCl₃/TMS): δ2.07 (s, 3H), 2.19 (s, 12H), 4.30 (dd, J=1.4, 1.4 Hz, 4H), 4.52 (dd, J=1.4, 1.4 Hz, 2H), 5. 26 (m, 3H), 5. 43 (m, 3H), 6.10 (m, 3H), 6. 67 (s, 4H), 6.80 (d, J=9.0 Hz, 2H), 6.96 (d, J=9.0 Hz, 2H).

### <Heat Resistance Evaluation>

To compare the heat resistance of compounds (p-3), (p-14), and (p-17) obtained according to the present invention with that of triallyl isocyanurate widely used as a curing agent, the 5% weight loss temperature of each compound was measured using the following apparatus and conditions. In addition, the onset temperature (Tm) (C) of compounds (p-3), (p-14), and (p-17) according to the present invention was measured. The onset temperature here refers to a temperature at an intersection of a tangent line at an inflection point on the low-temperature side on a DSC endothermic peak and an extension of a baseline.

The measurement results are shown in Table 1. (5% weight loss temperature measurement)

Apparatus: DTG-60A/manufactured by Shimadzu Corporation

Temperature: 30° C.→400° C. (heating rate, 10° C./min)

Measurement atmosphere: open; nitrogen, 50 mL/min

Sample weight: 8 to 12 mg

Sample container material: aluminum

TABLE 1

| | 5% weight loss | |
|---|---|---|
| Sample | temperature (° C.) | Tm (° C.) |
| Compound (p-3) | 237 | 58.7 |
| Compound (p-14) | 223 | 71.6 |
| Compound (p-17) | 238 | 78.3 |
| Triallyl isocyanurate | 176 | — |

As shown in Table 1, it has become clear that the tris(allyl ether) compounds according to the present invention having a triphenylalkane backbone have higher heat resistance than triallyl isocyanurate.

In a situation of high-temperature heating at about 200° C. for curing a thermosetting resin forming a prepreg, triallyl isocyanurate undergoes a weight loss of 5% or more and thus has been confirmed to suffer from a problem of volatilization of components. On the other hand, it has also become clear that the compounds according to the present invention have high heat resistance and thus can contribute to reducing the problem of volatilization of components.

For compound (p-14) and compound (p-17), whose chemical structures differ only in the presence of a methyl group at the central carbon atom, it has also become clear that compound (p-17) having a methyl group, as compared to compound (p-14) not having a methyl group, has a 15° C. higher 5% weight loss temperature and an about 6° C. higher onset temperature, and has higher heat resistance.

### <Comparison of Claisen Rearrangement Reactivity Upon Heating (Hydroxyl Value Measurement)>

Using compounds (p-3), (p-14), and (p-17) according to the present invention and comparative compounds 1 and 2 above as samples, a test for evaluating Claisen rearrangement reactivity was performed.

A test tube containing accurately weighed 1 g of each sample was heated for 1 hour under temperature conditions of 200° C. assuming a heating temperature at the time when the sample was used as a curing agent for modified polyphenylene ether (m-PPE). A test tube for a blank without a sample was also provided, and for the heated sample, an unheated sample, and the blank, measurements were made in accordance with the hydroxyl value measurement (potentiometric titration) in JIS K 0070. Specifically, the following “measurement method” was used. The measurement results are shown in Table 2.

### (Measurement Method)

Ten grams of acetic anhydride was dissolved in pyridine to prepare 100 g of a solution, and exactly 10 mL of the solution was added to each of the above test tubes and stirred at 105° C. for 1 hour. Thereafter, the solution was cooled, and 10 g of ultrapure water and 30 g of ethanol were added. The solution was transferred to a beaker and adjusted using ethanol to a total volume of about 100 mL. While the solution was stirred, a 0.5 N ethanolic potassium hydroxide solution was added dropwise to determine the inflection point of a pH curve. For the titration, a potentiometric titrator (AT-510) manufactured by Kyoto Electronics Manufacturing Co., Ltd. was used.

The hydroxyl values (unit: KOHmg/g) before and after heating of each sample were calculated by the following formula (I), and the difference between the hydroxyl values was determined. In addition, the hydroxyl amounts before and after heating of each sample converted per mole of substrate were calculated by the following formula (II), and the difference between the hydroxyl amounts was determined.

Hydroxyl value (KOHmg/g)={56.1×0.5×(b−a)×F}/S (I)

Hydroxyl amount (mol)=[{0.5×(b−a)×F×M}/1000]/S (II)

S: Amount of sample collected (g)

a: Consumption of 0.5 N ethanolic potassium hydroxide solution (mL)

b: Consumption of 0.5 N ethanolic potassium hydroxide solution of blank (mL)

F: Titer of 0.5 N ethanolic potassium hydroxide solution

M: Molecular weight of each sample

TABLE 2

| | Difference | Hydroxyl |
|---|---|---|
| | in hydroxyl | amount per |
| | value | mole of sample |
| Sample | (KOHmg/g) | (mol) |
| Compound (p-3) | 103.0 | 0.91 |
| Compound (p-14) | 129.0 | 0.98 |
| Compound (p-17) | 75.1 | 0.65 |
| Comparative compound 1 | 383.0 | 2.82 |
| Comparative compound 2 | 316.1 | 2.38 |

As shown in Table 2, it has become clear that compounds (p-3), (p-14), and (p-17) according to the present invention have low Claisen rearrangement reactivity since the difference in hydroxyl value before and after heating and the difference in hydroxyl amount per mole of sample are smaller than those of comparative compounds 1 and 2 known in the art.

In comparison of compound (p-14) with compound (p-17), it has also become clear that compound (p-17) having a methyl group at the central carbon atom has even lower Claisen rearrangement reactivity than compound (p-14) not having a methyl group.

### <Dielectric Properties Evaluation>

Among the dielectric properties of a material, the dielectric constant (ε), according to Maxwell's equations, is related to the refractive index (n) of the material by “ε=n²” (this relation is described in, for example, Shogo Saito, Electrophotography, Vol. 11, No. 1, pp. 26-32, 1972). That is to say, materials having smaller refractive indices have lower dielectric constants.

Using compounds (p-3), (p-14), and (p-17) according to the present invention and comparative compounds 1 and 2 as samples, the refractive index was measured according to the following measurement method. In addition, the refractive index was measured after heating for 1 hour under temperature conditions of 130° C. and 200° C. assuming heating at the time when the sample was used as a curing agent for modified polyphenylene ether (m-PPE), and the change in refractive index was evaluated.

### (Measurement Method)

N-methylpyrrolidone and each sample were mixed at any weight ratio, and the refractive index of each solution at 20° C. was measured. For the measurement of the refractive index, an RA-500 refractometer manufactured by Kyoto Electronics Manufacturing Co., Ltd. was used. The refractive index values obtained and the concentrations of the solution were plotted to construct a linear function. In the function constructed, the value at the point where the sample concentration was 100% was determined by extrapolation, and this value was used as the refractive index of each sample alone.

The refractive indices of the samples before and after heating are as shown in Table 3.

TABLE 3

| | Before | After heating | After heating |
|---|---|---|---|
| Sample | heating | at 130° C. | at 200° C. |
| Compound (p-3) | 1.572 | 1.574 | 1.593 |
| Compound (p-14) | 1.580 | 1.581 | 1.638 |
| Compound (p-17) | 1.581 | 1.581 | 1.583 |
| Comparative compound 1 | 1.586 | 1.590 | 1.612 |
| Comparative compound 2 | 1.596 | 1.597 | 1.605 |

As shown in Table 3, it has become clear that compounds (p-3), (p-14), and (p-17), which are specific examples of the inventive compound, have lower refractive indices than comparative compounds 1 and 2 known in the art before heating and also after heating at 130° C.

In addition, it has become clear that compounds (p-3) and (p-17) have lower refractive indices than comparative compounds 1 and 2 also after heating at 200° C.

Furthermore, the change in refractive index of compound (p-17) from before heating to after heating at 200° C. is very small as 0.002, demonstrating that the change in refractive index upon heating can be suppressed.

From this, it has become clear that the compounds according to the present invention, although having dielectric constants lower than those of comparative compounds 1 and 2 known in the art, contribute to improving the dielectric properties of materials when the compounds according to the present invention are used as curing agents.

It seems that the reason why the compounds according to the present invention have lower refractive indices than the comparative compounds also after heating is because the Claisen rearrangement reactivity upon heating is low, and the hydroxyl amount, which is a factor of increase in dielectric constant, is small.

The small change in refractive index of compound (p-17) before and after heating is presumably because the presence of a methyl group at the central carbon atom further inhibited the Claisen rearrangement reactivity, in view of the results of the above hydroxyl value measurement.

1. A tris(allyl ether) compound represented by general formula (1): 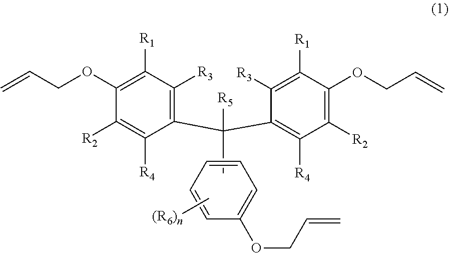
wherein R₁ and R₆ each independently represent a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms, R₂, R₃, and R₄ each independently represent a hydrogen atom, a linear or branched alkyl group having 1 to 6 carbon atoms, a cyclic alkyl group having 3 to 6 carbon atoms, a linear or branched alkoxy group having 1 to 6 carbon atoms, or a cyclic alkoxy group having 3 to 6 carbon atoms, R₅ represents a hydrogen atom or a linear or branched alkyl group having 1 to 6 carbon atoms, and n represents 0 or an integer of 1 to 4.

2. The tris(allyl ether) compound according to claim 1, represented by general formula (2): 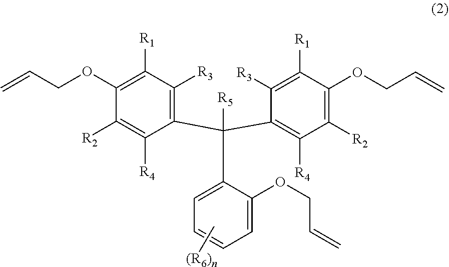
wherein R₁ to R₆ and n are as defined in general formula (1).

3. The tris(allyl ether) compound according to claim 1, represented by general formula (3): 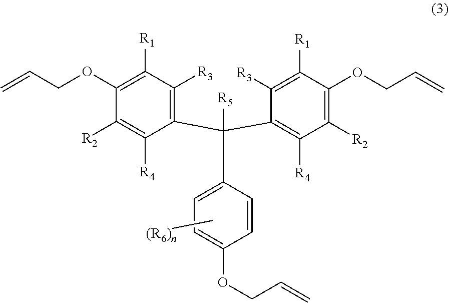
wherein R₁ to R₆ and n are as defined in general formula (1).
